(19) United States
(12) Patent Application Publication
(10) Pub. No.: US 2019/0105016 A1
(43) Pub. Date: Apr. 11, 2019

(54) SYSTEM AND METHOD FOR ULTRASOUND IMAGING WITH A TRACKING SYSTEM

(71) Applicant: General Electric Company, Schenectady, NY (US)

(72) Inventors: Julio Jenaro, Salzburg (AT); Reinhold Bruestle, Frankenburg (AT); Manuel Schoenauer, Thalgau (AT); Andreas Kremsl, St. Wolfgang (AT); Marco Brusaca, Attersee am Attersee (AT)

(21) Appl. No.: 15/725,852

(22) Filed: Oct. 5, 2017

(51) Int. Cl. A61B 8/00

(52) U.S. Cl. CPC A61B 8/4254; A61B 8/463

(57) ABSTRACT

A medical imaging system and a method of ultrasound imaging with a tracking system includes identifying a volume-of-interest from a first ultrasound image acquired from a first position and orientation, tracking the probe using the tracking system as the probe is moved to a second position and orientation, calculating an orientation adjustment that should be applied to the probe from the second position and orientation to bring the volume-of-interest within a field-of-view of the probe, and displaying both a tilt graphical indicator and a rotation graphical indicator on a display device to illustrate the orientation adjustment.

### FIELD OF THE INVENTION

This disclosure relates generally to a system and method for ultrasound imaging with a tracking system. The system and method includes displaying both a tilt graphical indicator and a rotation graphical indicator on a display device to indicate how a probe needs to be adjusted in order to image a volume-of-interest.

### BACKGROUND OF THE INVENTION

Current ultrasound imaging protocols often require a clinician to scan an ultrasound volume-of-interest from different positions and orientations. For instance, it is common to acquire images of a fetal heart from multiple different positions and orientations. However, it can be difficult, even for a skilled user, to correctly orient the probe in order to acquire images of the desired volume-of-interest from the different positions. The patient's anatomy looks different from various perspectives and there are many degrees of freedom (position, rotation, and tilt) for adjusting the probe. The difficulty in locating and scanning the desired volume-of-interest from different probe positions may make it difficult or impossible for an inexperienced user to complete the protocol and may result in a longer total scan time even for an experienced user to complete the protocol.

For these and other reasons an improved medical imaging system and method for providing feedback instructing a user how to adjust an orientation of the probe to image the volume-of-interest is desired.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes identifying a volume-of-interest using a first ultrasound image acquired with a probe from a first position and orientation, tracking the probe using a tracking system as the probe is moved from the first position and orientation to a second position and orientation, calculating with a processor, an orientation adjustment that should be applied to the probe from the second position and orientation to bring the volume-of-interest within a field-of-view of the probe based on the tracking the probe, and displaying both a tilt graphical indicator and a rotation graphical indicator on a display device to illustrate the orientation adjustment.

In an embodiment, a method of ultrasound imaging includes positioning a probe in a first position and a first orientation, acquiring a first ultrasound image with the probe while the probe is in the first position and the first orientation, selecting a volume-of-interest from the first ultrasound image, and moving the probe from the first position and the first orientation to a second position and a second orientation. The method includes tracking the probe with a tracking system as the probe is moved from the first position and the first orientation to the second position and the second orientation, calculating, with a processor, an orientation adjustment of the probe to position the volume-of-interest within a field-of-view of the probe while the probe is in the second position, and displaying both a tilt graphical indicator and a rotation graphical indicator on a display device to illustrate the orientation adjustment for the probe that was calculated by the processor.

In an embodiment, a medical imaging system includes an ultrasound imaging system including a probe, a display device, and a processor in electronic communication with the probe and the display device. The medical imaging system includes a tracking system in electronic communication with the processor, where the tracking system is configured to provide position and orientation data for the probe. Where the processor is configured to control the probe to acquire a first ultrasound image with the probe in a first position and orientation, receive a selection of a volume-of-interest based on the first ultrasound image, calculate the position of the volume-of-interest based on the position and orientation data from the tracking system, calculate an orientation adjustment for the probe with the probe at a second position and orientation that is different than the first position and orientation based on the position and orientation data from the tracking system, where the orientation adjustment represents a change in orientation from the second position and orientation that should be applied to the probe to bring the volume-of-interest within a field-of-view of the probe, and display both a tilt graphical indicator and a rotation graphical indicator on the display device to illustrate the orientation adjustment.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an medical imaging system in accordance with an embodiment;

FIG. 2 is a schematic diagram of a tracking system in accordance with an embodiment;

FIG. 3 is a schematic representation of a coordinate axis oriented with respect to a probe in accordance with an embodiment;

FIG. 4 is a flow chart of a method in accordance with an embodiment;

FIG. 5 is a schematic representation of a patient and a probe in accordance with an embodiment;

FIG. 6 is schematic representation of a probe, a volume-of-interest, and a coordinate axis in accordance with an embodiment;

FIG. 7 is a schematic representation of a screenshot in accordance with an embodiment;

FIG. 8 is a schematic representation of a screenshot in accordance with an embodiment; and

FIG. 9 is a schematic representation of a screenshot in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

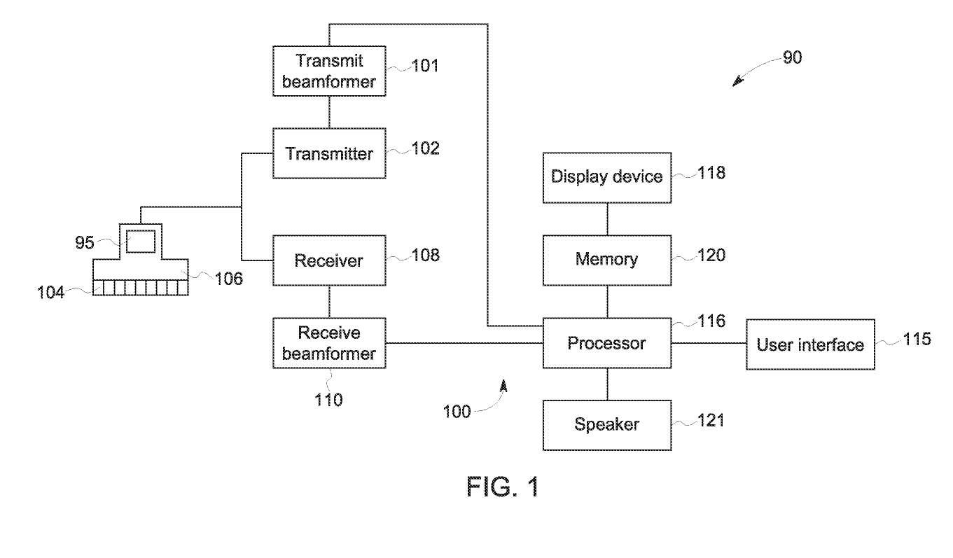

FIG. 1 is a schematic diagram of a medical imaging system 90 in accordance with an embodiment. The medical imaging system 90 includes a tracking system 95 and an ultrasound imaging system 100 in accordance with an embodiment. The ultrasound imaging system 100 includes a transmit beamformer 101 and a transmitter 102 that drive elements 104 within a probe 106 to emit pulsed ultrasonic signals into a body (not shown). The probe 106 may be any type of probe, including a linear probe, a curved array probe, a 1.25D array, a 1.5D array, a 1.75D array, or 2D matrix array probe according to various embodiments. The probe 106 may also be a mechanical 3D probe including one or more arrays of elements and a mechanism that causes the one or more arrays of elements to tilt or “wobble” in order to acquire a volume of data. Preferably, the probe 106 may be a 2D matrix array probe or a mechanical 3D probe that that is configured for acquiring ultrasound data from a volume-of-interest. However, other embodiments may use a 2D probe and the tracking system 95 in order to acquired ultrasound data from a volume-of-interest.

4D ultrasound data contains information about how a volume changes over time. Each of the volumes may include a plurality of 2D images or slices. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, and parameters, and the like. The user interface 115 may include one or more user input devices such as a keyboard, hard keys, a touch pad, a mouse, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The beamformer 110 may be configured to perform conventional beamforming techniques as well as techniques such as retrospective transmit beamforming (RTB). The processor 116 is in electronic communication with the user interface 115, the memory 120, the display device 118, the transmit beamformer, the receive beamformer 110 and the tracking system 95. The processor 116 may be in electronic communication with the user interface 115, the memory 120, the display device 118, the transmit beamformer, the receive beamformer 110 and the tracking system 95 through wired or wireless techniques. The ultrasound imaging system 100 may optionally include a speaker 121 controlled by the processor 116.

The tracking system 95 includes an accelerometer 128, a gyroscope 129, and a magnetometer 132 according to an embodiment. Other embodiments may include a tracking system with an accelerometer and a gyroscope, but without a magnetometer. The accelerometer 128 is a component adapted to measure acceleration. The accelerometer 128 may include one or more of a piezoelectric component, a piezoresistive component and a capacitive component in order to convert acceleration into an electrical signal. The accelerometer 128 may be a micro electro-mechanical system (MEMS) according to an embodiment. The gyroscope 129 may include a spinning wheel or disc to determine changes in angular orientation. According to other embodiments, the gyroscope may be a vibrating structure gyroscope that includes a vibrating structure to determine any changes in angular orientation. The vibrating structure gyroscope may, for instance be manufactured using microelectromechanical systems (MEMS) technology. The magnetometer 132 may include a magnetized component or a plurality of coils that are sensitive to an external magnetic field. The magnetometer 132 is configured to output signals indicating the orientation of the magnetometer 132 with respect to the external magnetic field. The magnetized component or the plurality of coils within the magnetometer 132 may detect the orientation of the external magnetic field, which is used, in turn, to determine the orientation of the magnetometer 132 with respect to the external magnetic field.

The external magnetic field may be due to the earth's magnetic field or the external magnetic field may be due to the combination of the earth's magnetic field and the contribution of any local magnetic field sources. For example, according to some embodiments, the tracking system 95 may include a magnetic field generator. The magnetometer may be used to compensate for drift within one or both of the accelerometer 128 and the gyroscope 129. For instance, the gyroscope 129 is very sensitive to small changes in angular momentum, but may be susceptible to drift. The magnetometer 132 provides information regarding the orientation of the probe 106 with respect to the external magnetic field (such as orientation of the probe with respect to the cardinal directions: North, South, East, and West) and the magnetometer may also provide information regarding the horizontality of the gyroscope. For instance, the signals from the magnetometer 132 may be used to determine the tilt of the gyroscope with respect to a plane defined by the North, South, East, and West directions. The signals from the magnetometer 132 may be used to calibrate the horizontality of the gyroscope 129 and reduce the amount of uncertainty in the gyroscope 129 due to drift. According to some embodiments, signals from the magnetometer 132 may also be used to determine the absolute position of the probe 95, which in turn, may be used to compensate for drift within the accelerometer 128.

The processor 116 receives the signals, including position and orientation data, from the tracking system 95 and processes the signals to determine the position and orientation of the probe 106. For instance, the processor 116 may integrate signals from the gyroscope 130 from an initial position to determine changes in a tilt and rotation of the probe 95. Likewise, the processor 116 may integrate signals from the accelerometer 128 from an initial position in order to determine the change in position of the probe 95. The processor 116 may use signals from the magnetometer 132 to compensate for drift within the accelerometer 128 and/or to initialize the position of the probe 95 with respect to the external magnetic field. The processor 116 may establish the position of the coordinate axis anywhere, but according to an embodiment, the coordinate axis 130 may be oriented with respect to the probe 106 at an initial position. The user may, for instance, press a button or control on the user interface 115 to determine the position of the coordinate axis 130 or to determine an initial position and orientation of the probe 106. According to another embodiment, the processor 116 may automatically position an origin of the coordinate axis 130 in response to acquiring an image. According to an embodiment, the processor 116 may align the coordinate axis 130 with the probe 106. An example showing the coordinate axis 130 aligned with the probe is shown in FIG. 3, which will be described hereinafter. The processor 116 may also use signals from the magnetometer 132 to position a coordinate axis 130. The coordinate axis 130 includes an x-axis 133, a y-axis 134, a z-axis 136, and an origin 138. According to an embodiment, the origin 138 may be positioned in the center of the probe 106 or in the center of a lens of the probe 106 that would be in contact with the patient. According to an embodiment, the x-axis 132 may be aligned with an azimuth direction 140 of the probe 106, the y-axis 134 may be aligned with an elevation direction 142 of the probe 106, and the z-axis 136 may be aligned with a depth direction 144 of the probe 106.

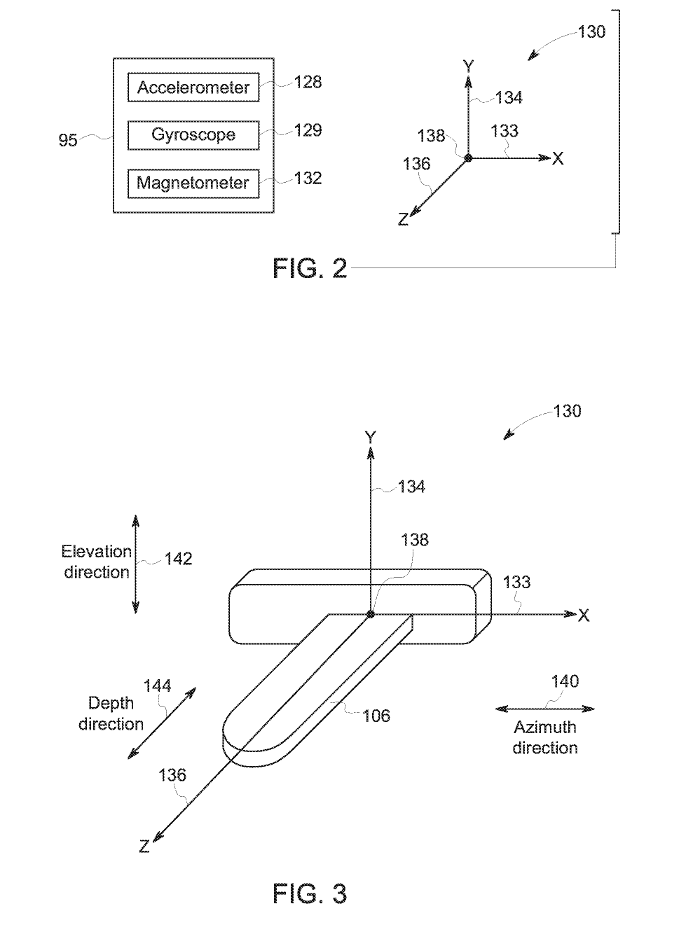

FIG. 2 is a schematic representation of the tracking system 95 in accordance with an embodiment. The tracking system 95 includes the accelerometer 128, the gyroscope 129, and the magnetometer 132 as described hereinabove. FIG. 2 also includes a coordinate axis 130. The coordinate axis 130 includes an x-axis 133, a y-axis 134, a z-axis 136 and an origin 138. The position of the coordinate axis 130 may be set at a preset location or the processor 116 may position the coordinate axis 130 at a position indicated through a user input, entered through the user interface 115 or based on the position of the probe 106 while one or more images are acquired. According to an embodiment, the processor 116 may position the coordinate axis 130 relative to a portion of the probe 106 at the position where a first ultrasound image is acquired, as will be described in additional detail with respect to FIG. 3.

FIG. 3 shows a representation of the coordinate axis 130 oriented with a schematic representation of a probe 106 in accordance with an embodiment. The x-axis 133 is aligned with an azimuth direction 140 of the probe, the y-axis 134 is aligned with an elevation direction 142 of the probe 106, and the z-axis 136 is aligned with a depth direction 144 of the probe 106. The origin 138 of the coordinate axis 130 may be positioned in the center of an array as shown in FIG. 3 with the probe 106 at an initial position. The user may, for instance, select the initial position which would set the position of the coordinate axis 130. In FIG. 3, the z-axis 136 of the probe 106 coincides with a longitudinal axis of the probe 106. In other embodiments, the user may set an initial position and orientation of the probe 106 with respect to a coordinate axis that is not aligned with the probe 106. According to other embodiments, the processor 116 may automatically store the position and orientation of the probe 106 with respect to the coordinate axis 130 at every position and orientation from which an image is acquired.

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with a display device 118, and the processor 116 may process the ultrasound data into images for display on the display device 118. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU) or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. Real-time frame or volume rates may vary based on the size of the region or volume from which data is acquired and the specific parameters used during the acquisition. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to display as an image. It should be appreciated that other embodiments may use a different arrangement of processors. For embodiments where the receive beamformer 110 is a software beamformer, the processing functions attributed to the processor 116 and the software beamformer hereinabove may be performed by a single processor such as the receive beamformer 110 or the processor 116. Or, the processing functions attributed to the processor 116 and the software beamformer may be allocated in a different manner between any number of separate processing components.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may be a tangible and non-transitory computer readable medium such as flash memory, RAM, ROM, EEPROM, and/or the like.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

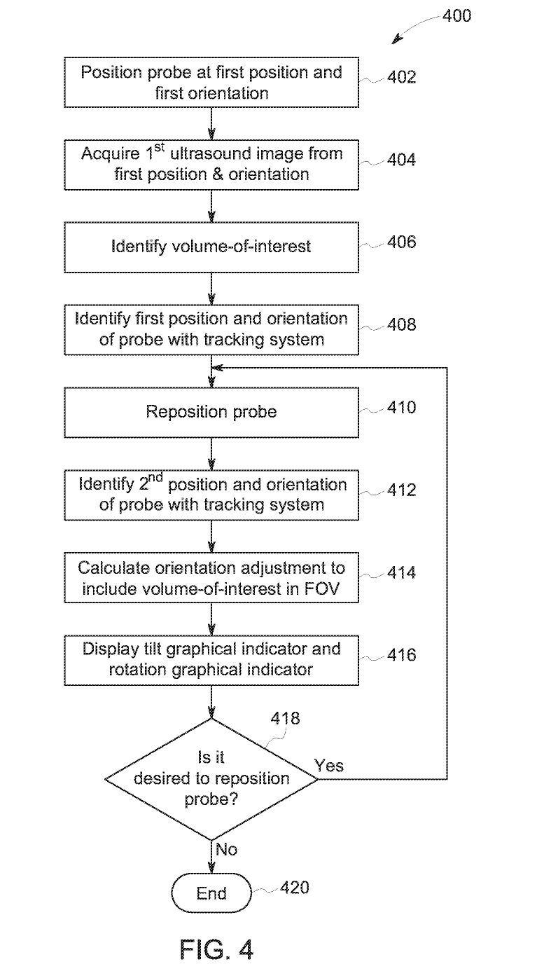

FIG. 4 is a flow chart of a method 400 in accordance with an exemplary embodiment. The individual blocks of the flow chart represent steps that may be performed in accordance with the method 400. Additional embodiments may perform the steps shown in a different sequence and/or embodiments may include additional steps not shown in FIG. 4. The technical effect of the method 400 is the calculation of an orientation adjustment, and the display of a tilt graphical indicator and a rotation graphical indicator to help the user position the probe 106 to acquire a previously identified volume-of-interest. The method 400 will be described according to an embodiment using the medical imaging system 90 shown in FIG. 1.

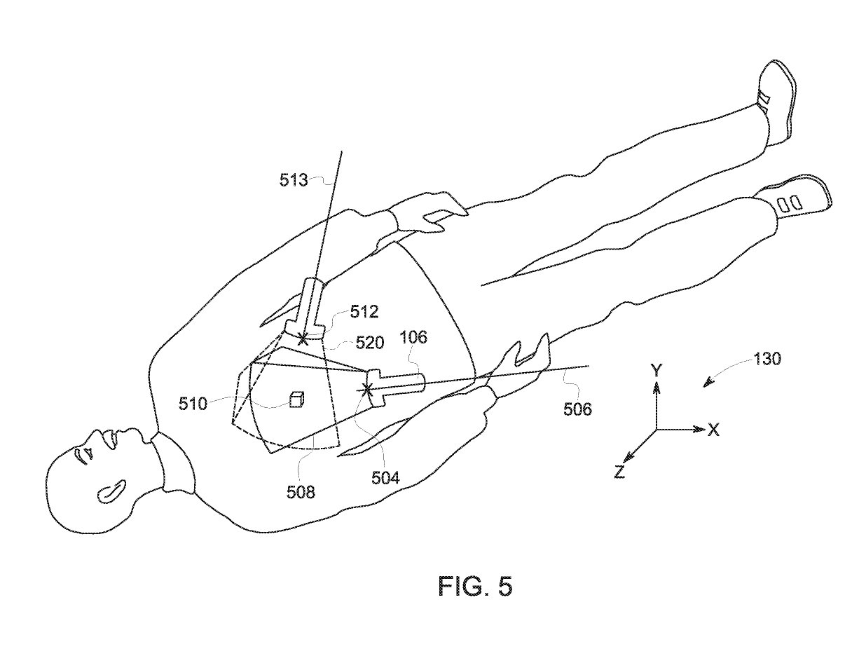

FIG. 5 is a schematic representation of a patient and a probe. Referring to FIGS. 4 and 5, at step 402, a clinician positions the probe 106 at a first position 504 and a first orientation. In this application, when referring to the ultrasound probe 106, the term “position” will be defined to include the spatial location of a point on the probe 106. In this application, when referring to the ultrasound probe 106, the term “orientation” will be defined to include the tilt and rotation of the probe at a specific location. The direction of a vector positioned along the longitudinal axis of the probe 106 may be used to represent the tilt of the probe 106. The first position 504 is represented by an “X” on the surface of the patient where a center of the transducer array of the probe 106 is located. The first tilt is represented by line 506. It should be appreciated that the position of the probe 106 may be determined with respect to any other part of the probe 106 according to other embodiments. The first position 504 of the probe 106 is determined with respect to the coordinate axis, such as the coordinate axis 130 that is schematically represented in FIG. 5. In this disclosure, the term “position” refers to the position of the object (such as the probe 106) in three-dimensional space with respect to a coordinate axis 130. The term “orientation” refers to the tilt and rotation of the object (such as the probe 106) with respect to the coordinate axis 130. The probe 106 could, for instance, be in many different orientations while at the same position. Likewise, the probe 106 could have the same orientation with respect to the coordinate axis 130 in many different positions.

At step 404, the processor 116 controls the probe 106 to acquire a first ultrasound image from the first position 504 and the first orientation. In this disclosure, the phrase “first position and orientation” has the same meaning as the phrase “first position and first orientation” Likewise, the phrase “second position and orientation” has the same meaning as the phrase “second position and second orientation”. The probe 106 has a field-of-view (FOV) 508. The FOV 508 represents the area or volume from which the probe 106 can acquire an ultrasound image while in a given position and orientation. For example, the FOV 508 represents the volume from which the probe 106 can acquire an image while in the first position 504 and the first orientation. The first ultrasound image includes information from within the first field-of-view 508.

At step 406, the processor 116 identifies a volume-of-interest 510 from within the field-of-view 508. According to an embodiment, the processor 116 may select the volume-of-interest (VOI) 510 in response to user input entered through the user interface 115. For example, the user may use a trackball, a mouse, a touchscreen or other user interface control to select a sub-volume from within the field-of-view 508 as the volume-of-interest 510. The user may, for instance, select a region-of-interest (i.e., a 2D region) from a 2D image generated from ultrasound data. The user may than input commands to select a thickness of the 2D region in a direction perpendicular to the plane of the 2D image. By adjusting this thickness, the user may select the volume-of-interest, which is then identified by the processor 116. It should be appreciated, that the user may select the volume-of-interest in other ways. For instance, the user may identify the volume-of-interest from a volume rendering, or the user may identify the volume-of-interest by positioning a geometric shape, such as a cube, sphere, or other shape, on either a 2D image or a volume rendering in order to select the volume-of-interest.

At step 408, the processor 116 identifies a first position 504 and the first orientation of the probe 106 based on position and orientation data from the tracking system 95. First line 506 represents the first tilt of the probe 106. The processor 116 may store the first position 504 and the first orientation of the probe 106 in a memory or storage such as the memory 120. The user may select a position/orientation of the probe 106, such as with an input form the user interface 115, or the processor 116 may automatically store the position and orientation of the probe 106 used during the acquisition of one or more images. The processor 116 identifies the first position and the orientation of the probe 106 in order to calculate the position of the volume-of-interest 510 with respect to the probe 106 in the first position and orientation.

At step 410, the clinician repositions the probe 106 with respect to the patient 502. According to an exemplary embodiment, the clinician may move the probe 106 from the first position 504 and the first orientation to a second position 512 and a second orientation. A second line 513 represents a second tilt of the probe 106. For many protocols, it is necessary to image the volume-of-interest 510 from multiple different directions. For example, when imaging a fetal heart, it is often necessary to obtain images of the volume-of-interest from multiple different probe positions. The clinician may reposition the probe 106 in order to get a better view of some of the structure within the volume-of-interest 510.

At step 412, the processor 116 identifies the second position 512 and a second orientation of the probe 106 based on position and orientation data from the tracking system 95. As discussed above, the clinician may set the position of the coordinate axis 130 based on the first position 504 and first orientation of the probe 106. According to other embodiments, the processor 116 may store first position and orientation of the probe 106 with respect to the coordinate axis 130 in the memory in response to acquiring the first image. At step 412, the processor 116 identifies the second position 512 and the second orientation of the probe 106 based on position and orientation data from the tracking system 95. As described previously, the processor 116 may, for instance, integrate signals from the gyroscope 129 to determine a change in orientation, integrate signals from the accelerometer 128 to determine a change in position and use signals from the magnetometer 132 to compensate for drift and/or use the signals from the magnetometer 132 to confirm the position of the probe 106 with respect to an external magnetic field.

At step 414, the processor 116 calculates an orientation adjustment that need to be applied to the probe 106 (from the second position 512 and orientation) in order to include the volume-of-interest 510 within the field-of-view of the probe 106 while the probe is in the second position 512. As described above, the processor 116 determines the change in position and orientation of the probe 106 from the first position 504 and orientation to the second position 512 and orientation based on the position and orientation data from the tracking system 95. The processor 116 calculates the position of the volume-of-interest 510 with respect to the coordinate axis 130 based on the position of the volume-of-interest 510 with respect to the probe 106. The processor 116 may, for instance, rely on the depth of the volume-of-interest 510 and the azimuthal and elevational positioning of the VOI 510 with respect to the probe 106 in order to calculate the position of the VOI 510 with respect to the coordinate axis 130.

The processor 116 calculates a change in orientation that must be applied to the probe 106 in order to include the VOI 510 within the field-of-view 520 while the probe 106 is in the second position 512.

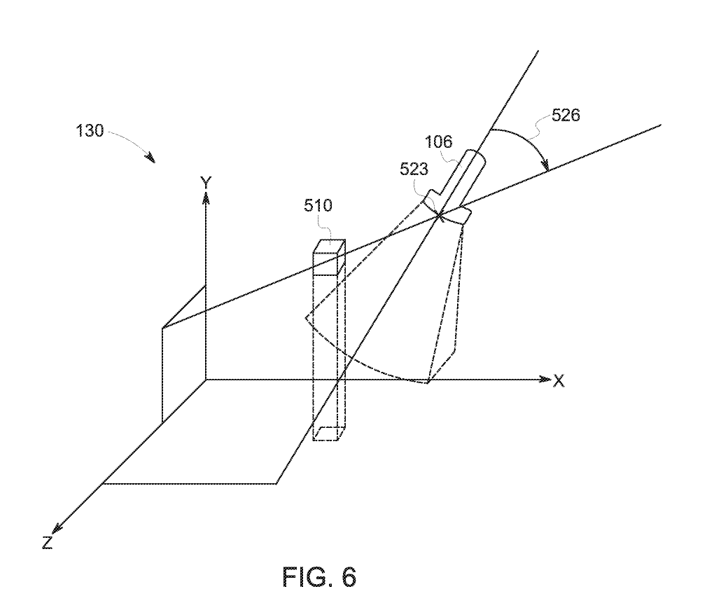

FIG. 6 is a schematic representation of the probe 106, the VOI 510 and the coordinate axis 130. FIG. 6 also includes the field-of-view 520 of the probe 106. According to an embodiment, the coordinate axis 130, as represented in FIG. 6, may be positioned to correlate with a first position and orientation of the probe 106. A line 522 represents a tilt of the probe 106 at the point 523, while line 524 represents a desired orientation of the probe 106 at the point 523 in order to include the VOI 510 within the field-of-view. Arrow 526 represents the change in tilt that must be applied to the probe 106 in order to orient the longitudinal axis of the probe 106 along the line 524. While not shown, the change in orientation may include a rotation adjustment of the probe 106 in addition to a tilt adjustment.

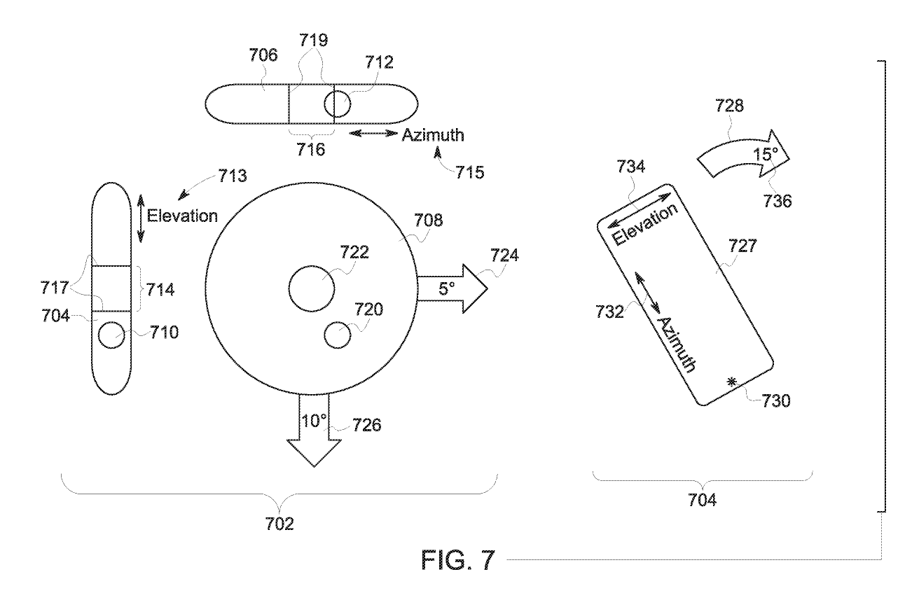

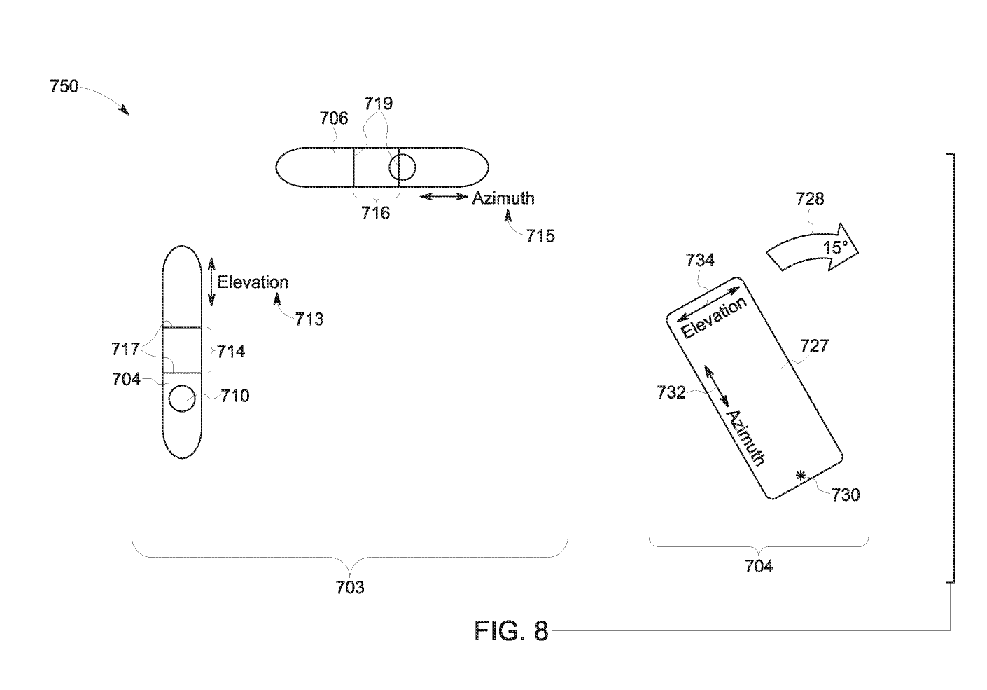

FIG. 7 is a schematic representation of a screenshot that would be displayed on the display device 118. FIG. 7 includes a tilt graphical indicator 702 and a rotation graphical indicator 704.

According to an exemplary embodiment, the tilt graphical indicator 702 includes a first virtual spirit level 704, a second virtual spirit level 706 and a virtual circular spirit level 708. The first virtual spirit level 704 is disposed at a 90 degree angle to the second virtual spirit level 706. As shown in FIG. 7, the first virtual spirit level 704 is disposed in a vertical direction on the display device 118 and the second virtual spirit level 706 is disposed in a horizontal direction on the display device 118. According to an embodiment, the first virtual spirit level 704 may represent the tilt needed in an elevation direction 734 and the second virtual spirit level 706 may represent the tilt needed in an azimuth direction 732. For example, the tilt graphical indicator may optionally include a first label 713 to indicate that the first virtual spirit level 704 represents the elevation direction and a second label 715 to indicate that the second virtual spirit level 706 indicates the azimuthal direction. Collectively, the first virtual spirit level 704 and the second virtual spirit level 706 provide enough information to instruct the clinician how to adjust the tilt of the probe 106.

The tilt graphical indicator 702 shown in FIG. 7 also includes the virtual circular spirit level 708. The virtual circular spirit level 708 includes a third virtual bubble 720 and a bullseye 722. The virtual circular spirit level 708 is a virtual representation of a conventional circular spirit level. The virtual circular spirit level emulates a conventional circular spirit level. The virtual circular spirit level 708 provides information to the user regarding how to tilt the probe 106 to image the volume-of-interest 510 from the current probe position, which may be the second probe position 512 (shown in FIG. 5) according to an embodiment. The tilt graphical indicator 702 also includes a first arrow 724 and a second arrow 726. The first arrow 724 indicates the amount the probe 106 needs to be tipped in the azimuth direction and the second arrow 726 indicates the amount the probe 106 needs to be tipped in the elevation direction in order to acquire an image including the VOI 510.

Each of the virtual spirit levels (i.e., the first virtual spirit level 704 and the second virtual spirit level 706) behaves like a conventional spirit level. A conventional spirit level is an instrument used for determining if a surface is horizontal (or vertical). A conventional spirit level typically includes a transparent vial that is mostly filled with a liquid. A bubble occupies the volume in the vial that is not filled with the liquid. The vial is either slightly curved or tapers in shape so it is widest at the mid-point and narrower at the ends. The center of the vial is typically marked with two lines. The bubble is always positioned at the highest point in the vial, and a user is able to tell when the conventional spirit level is either horizontal or vertical when the bubble is positioned between the two lines. The user can tell which way the spirit level needs to be tilted to position the spirit level in either a horizontal or a vertical orientation based on the position of the bubble with respect to the two lines. A conventional circular spirit level is typically an instrument with a flat bottom and a convex face made from a transparent material. The volume between the flat bottom and the convex face is incompletely filled with a fluid and the bubble is formed in the remaining volume. The bubble naturally rises to the highest point in the conventional circular spirit level. The conventional circular spirit level typically includes one or more circles, or bull's eye rings, to mark the center of the convex face. When the conventional circular spirit level is placed on a flat surface, the bubble will be in the center of the circle/bull's eye ring. The conventional circular spirit level can indicate how horizontal a surface is in multiple directions, whereas the conventional spirit level only indicates how horizontal/vertical a surface is in one direction. Conventional spirit levels are well-known by those skilled in the art and will therefore not be described in additional detail. The virtual spirit levels (704, 706 and 708) emulate the behavior of conventional spirit levels, but instead of indicating one of horizontal or vertical, the virtual spirit levels (704, 706, and 708) indicate when the probe 106 is in the proper orientation to acquire an image of the volume-of-interest. The virtual spirit levels show in FIG. 7 help the clinician position the probe 106 in the desired orientation for acquiring a previously identified volume-of-interest. Instead of indicating either vertical or horizontal, the virtual bubbles in the virtual spirit levels are centered within the pair of lines or marks when the probe is in the correct orientation to acquire an image including the volume-of-interest from a specified position. The goal for the clinician is to tilt the probe so that a first virtual bubble 710 is within a first desired zone 714 in the first virtual spirit level 704 and a second virtual bubble 712 is within a second desired zone 716 in the second virtual spirit level 706. The first desired zone 714 is indicated by a first pair of lines 717 and the second desired zone 716 is indicated by a second pair of lines 719.

As the tilt of the probe 106 is adjusted, the position of the first virtual bubble 710 and the second virtual bubble 712 both behave like a conventional spirit levels with respect to adjusting the orientation of the probe 106. In other words, tipping the probe 106 in the direction of the first virtual bubble 710 with respect to the first desired zone 714 in the elevation direction will cause the virtual bubble 710 to move in the direction of the first desired zone. Manipulating the tilt of the probe 106 until both virtual bubbles are in the respective desired zones will result in having the probe 106 with the correct tilt to image the volume-of-interest 510.

The rotation graphic indicator 704 includes a probe icon 727 and an arrow 728. The probe icon 727 represents a top view of the probe. The probe icon 727 may include a marker 730 that corresponds with a marker on the probe 106 to help the clinician stay orientated when viewing the rotation graphic indicator 704. Additionally, or instead of the marker 730, the probe 106 may include a first label 732 indicating an azimuth direction and a second label 734 indicating an elevation direction. It should be appreciated that in some embodiments, the probe icon 727 may not include one or more of the indicator 730, the first label 732, and the second label 734.

The arrow 728 indicates the direction that the user needs to rotate the probe 106 in order to image the volume-of-interest 510. The rotation graphic indicator 704 may include a number 736 indicating the number of degrees that that probe needs to be rotated in order to image the volume-of-interest 510. For instance, in the embodiment shown in FIG. 7, the probe 106 needs to be rotated 15 degrees in a clockwise direction.

At step 416, the processor 116 controls the display of both a tilt graphical indicator, such as the tilt graphical indicator 702, and a rotation graphical indicator, such as the rotation graphical indicator 704. The clinician may optionally reposition the probe 106 at step 418 of the method 400. If the user repositions the probe 106, the method 400 advances from step 418 to step 410, and steps 410, 412, 414, 416, and 418 are repeated. Steps 410, 412, 414, 416, and 418 may be iteratively repeated may times as the clinician fine tunes the position of the probe 106. It should be appreciated that the tilt graphical indicator 702 and the rotation graphical indicator 704 may be adjusted in real-time by the processor 116 as the orientation and/or the position of the probe 106 is adjusted. By adjusting tilt graphical indicator 702 and the rotation graphical indicator 704 in real-time, the processor 116 provides real-time feedback regarding the way the orientation of the probe 106 should be adjusted in order to image the volume-of-interest 510. Additionally, if the clinician should move the position of the probe 106, either by accident or deliberately, the processor 116 will adjust the tilt graphical indicator 702 and the rotation graphical indicator 704 in order to provide instructions based on the real-time position and orientation of the probe 106 to adjust the probe 106 in order to image the volume-of-interest 510.

Both the rotation graphical indicator 704 and the tilt graphical indicator 702 are linked to each other. In other words, as the rotation of the probe 106 is adjusted, the amount of tilt that needs to be applied to the probe in the azimuthal and elevation directions changes since the positions of the azimuthal and elevation directions have been modified with respect to the volume-of-interest 510 in the patient. As such, if the rotation of the probe is adjusted, the tilt graphical indicator 702 will be adjusted to reflect the tilt that needs to be applied to the probe from its current (i.e., real-time) position and orientation. The rotation graphical indicator 704 may likewise be adjusted as the tilt of the probe 106 is adjusted. Although it should be appreciated that if the user keeps the probe 106 in the same position as the tilt is adjusted, it may not be necessary for the processor 116 to adjust the desired rotation indicated by the rotation graphical indicator 704.

In some embodiments, the processor 116 may provide control signals that result in the playing of acoustic feedback through the speaker 121 either in addition to the rotation graphical indicator 704 and the tilt graphical indicator 702 or instead of the rotation graphical indicator 704 and the tilt graphical indicator 702. For example, the processor 116 may control the speaker 121 to emit a tone that provides acoustic feedback as the user is in the process of repositioning the probe 106. For instance, the processor 116 may alter one or more of a frequency of a tone, an amplitude of a tone, or a repetition interval of a series of tones to provide feedback when the user is moving the probe from the first position and orientation to the second position and orientation. According to an embodiment, the processor 116 may adjust the acoustic feedback so that the tone emitted through the speaker 121 increases in frequency (pitch) as the user moves the probe 106 closer to the second position and orientation and decreases in pitch as the user moves the probe 106 further away from the second position and orientation. According to an embodiment, the processor 116 may adjust the acoustic feedback so that the tone emitted through the speaker 121 increases in amplitude (volume) as the user moves the probe 106 closer to the second position and orientation and decreases in amplitude as the user moves the probe 106 further away from the second position and orientation. According to an embodiment, the processor 116 may emit a series of tones at a variable repetition interval. The processor 116 may adjust the repetition interval so that the series of tones emitted through the speaker has a shorter repetition interval as the user moves the probe 106 closer to the second position and orientation and has a longer repetition interval as the user moves the probe 106 further away from the second position and orientation. The acoustic feedback may be used to help guide the user to the correct second position and orientation for clinical situations where the user is not looking at the display device 114.

The processor 116 may use geometric calculations, such as trigonometry, to calculate and determine the position of the volume-of-interest with respect to the probe 106.

FIG. 8 is a schematic representation of a screenshot 750 in accordance with an embodiment. The screenshot 750 includes a tilt graphical indicator 703 and a rotation graphical indicator 704. Common reference numbers are used to identify identical elements that were previously described with respect to a prior figure. The rotation graphical indicator 704 includes a probe icon 727 and am arrow 728 and is identical to the rotation graphical indicator 704 described with respect to FIG. 7. The tilt graphical indicator 703 includes the first virtual spirit level 704 and the second virtual spirit level 706.

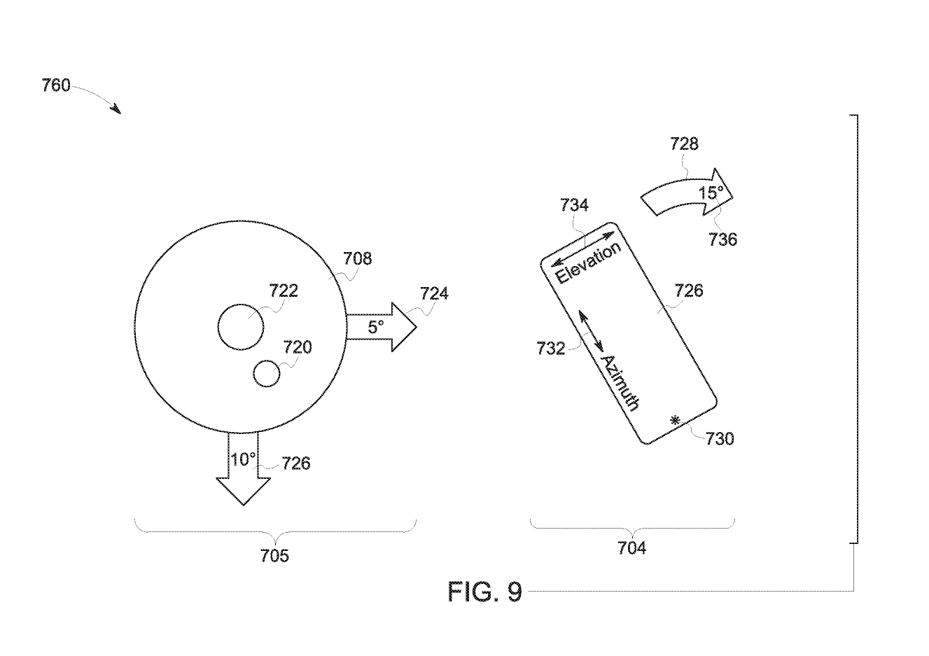

FIG. 9 is a schematic representation of a screenshot 760 in accordance with an embodiment. The screenshot 760 includes a tilt graphical indicator 705 and a rotation graphical indicator 704. Common reference numbers are used to identify identical elements that were previously described with respect to prior figures. The rotation graphical indicator 704 includes a probe icon 727 and an arrow 728 and is identical to the rotation graphical indicator 704 described with respect to FIG. 7. The tilt graphical indicator 703 includes the virtual circular spirit level 708.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
identifying a volume-of-interest using a first ultrasound image acquired with a probe from a first position and orientation;
tracking the probe using a tracking system as the probe is moved from the first position and orientation to a second position and orientation;
calculating with a processor, an orientation adjustment that should be applied to the probe from the second position and orientation to bring the volume-of-interest within a field-of-view of the probe based on said tracking the probe; and
displaying both a tilt graphical indicator and a rotation graphical indicator on a display device to illustrate the orientation adjustment.

2. The method of claim 1, wherein the tilt graphical indicator comprises a virtual spirit level.

3. The method of claim 1, wherein the tilt graphical indicator comprises a first virtual spirit level in a first direction and a second virtual spirit level in a second direction, where the second direction is perpendicular to the first direction.

4. The method of claim 3, wherein the first virtual spirit level represents an elevation direction and the second virtual spirit level represents an azimuth direction.

5. The method of claim 3, wherein the tilt graphical indicator further comprises a virtual circular spirit level.

6. The method of claim 1, wherein the tilt graphical indicator comprises a virtual circular spirit level.

7. The method of claim 1, wherein the rotation graphical indicator comprises a probe icon and an arrow indicating a rotation direction.

8. The method of claim 3, wherein the rotation graphical indicator comprises a probe icon and an arrow indicating a rotation direction.

9. The method of claim 5, wherein the rotation graphical indicator comprises a probe icon and an arrow indicating a rotation direction.

10. The method of claim 6, wherein the rotation graphical indicator comprises a probe icon and an arrow indicating a rotation direction.

11. The method of claim 6, wherein the tilt graphical indicator further comprises a first arrow indicating a first amount the ultrasound probe should be tilted in a first direction and a second arrow indicating a second amount the ultrasound probe should be tilted in a second direction.

12. A method of ultrasound imaging comprising:
positioning a probe in a first position and a first orientation;
acquiring a first ultrasound image with the probe while the probe is in the first position and the first orientation;
selecting a volume-of-interest from the first ultrasound image;
moving the probe from the first position and the first orientation to a second position and a second orientation;
tracking the probe with a tracking system as the probe is moved from the first positon and the first orientation to the second position and the second orientation;
calculating, with a processor, an orientation adjustment of the probe to position the volume-of-interest within a field-of-view of the probe while the probe is in the second position; and
displaying both a tilt graphical indicator and a rotation graphical indicator on a display device to illustrate the orientation adjustment for the probe that was calculated by the processor.

13. The method of claim 12, wherein the tilt graphical indicator comprises at least one virtual spirit level.

14. The method of claim 13, wherein the rotation graphical indicator comprises a probe icon and an arrow indicating a rotation direction.

15. The method of claim 14, wherein the rotation graphical indicator further comprises a number indicating the amount of degrees that the probe needs to be rotated in the rotation direction.

16. A medical imaging system comprising:
an ultrasound imaging system comprising a probe, a display device and a processor in electronic communication with the probe and the display device; and
a tracking system in electronic communication with the processor, where the tracking system is configured to provide position and orientation data for the probe;
wherein the processor is configured to:
control the probe to acquire a first ultrasound image with the probe in a first position and orientation;
receive a selection of a volume-of-interest based on the first ultrasound image;
calculate the position of the volume-of-interest based on the position and orientation data from the tracking system;
calculate an orientation adjustment for the probe with the probe in a second position and orientation that is different than the first position and orientation based on the position and orientation data from the tracking system, where the orientation adjustment represents a change in orientation from the second position and orientation that should be applied to the probe to bring the volume-of-interest within a field-of-view of the probe; and
display both a tilt graphical indicator and a rotation graphical indicator on the display device to illustrate the orientation adjustment.

17. The medical imaging system of claim 16, wherein the tracking system comprises an accelerometer.

18. The medical imaging system of claim 16, wherein the tracking system comprises a magnetometer.

19. The medical imaging system of claim 17, wherein the tracking system further comprises a gyroscope and a magnetometer.

20. The medical imaging system of claim 16, wherein the processor is configured to update the tilt graphical indicator and the rotation graphical indicator in real-time as the probe is being moved.
